(19) United States
(12) Patent Application Publication
(10) Pub. No.: US 2023/0183784 A1
(43) Pub. Date: Jun. 15, 2023

(54) QUADRUPLEX-BASED DETECTION OF A TARGET NUCLEIC ACID

(71) Applicant: Ohio State Innovation Foundation, Columbus, OH (US)

(72) Inventor: Besik KANKIA, Columbus, OH (US)

(21) Appl. No.: 17/924,639

(22) PCT Filed: May 13, 2021
(86) PCT No.: PCT/US2021/032178

§ 371 Date: Nov. 10, 2022

Related U.S. Application Data
(60) Provisional application No. 63/024,078, filed on May 13, 2020.

(51) Int. Cl. C12Q 1/6832

(52) U.S. Cl. CPC C12Q 1/6832; C12Q 1/6818

(57) ABSTRACT

Disclosed herein are oligonucleotides and methods for detecting a target nucleic acid, as well as methods of diagnosing a subject with a disease or a disorder by detecting a target nucleic acid using the oligonucleotides and methods disclosed herein. The oligonucleotides include a quadruplex-forming sequence. Prior to binding the target nucleic acid, the quadruplex-forming sequence is in a non-quadruplex conformation. Upon binding the target nucleic acid, the sequence transforms to the quadruplex conformation, which can be detected, for example, using optical methods. The methods are based on thermodynamic properties and do not require the use of enzymes. The methods can be used to diagnose a subject with a disease or a disorder, prognose the outcome of a disease or disorder, or predict the development of a future disease or disorder.

### RELATED APPLICATIONS

This application claims the benefit of U.S. Provisional Patent Application No. 63/024,078, filed May 13, 2020, which is hereby incorporated by reference in its entirety.

### FIELD

This invention relates to nucleic acid detection, and more particularly to non-enzymatic methods of nucleic acid detection and amplification.

### BACKGROUND

Due to self-assembly properties, nucleic acids have been established as excellent nanoscale materials to build various structures, devices and circuits. Nucleic acids can enable non-enzymatic signal amplification, which has great potential for the field of diagnostics. Non-enzymatic signal amplification reduces cost and enables point-of-care diagnostics, which can be useful in several scenarios (for example, in-field diagnostics or diagnosis of subjects in locations where highly technical medical machinery is unavailable). Non-enzymatic assays have greater stability and a longer shelf life at room temperature than enzymatic assays because they are based on oligonucelotides, which are more stable than enzymes.

The main challenge of the non-enzymatic assays is to design a spontaneous and sufficiently fast reaction in which a target, or catalyst, will trigger unidirectional structural reorganization of substrate molecules. The current non-enzymatic assays are based on strand-displacement reactions in DNA duplexes, which results into entropically or enthalpically more favorable product duplexes. However, these assays are thermodynamically favorable, with significant activity even in the absence of the catalyst. This activity in the absence of the target represents the main source for undesired background activity, also known as leakage. Improvements to decrease background activity are needed in the field of non-enzymatic nucleic acid detection and amplification.

### SUMMARY

Disclosed herein are oligonucleotides for use in non-enzymatic nucleic acid detection assays. The oligonucleotides include a first nucleotide sequence having a quadruplex-forming sequence, and a second nucleotide sequence operably linked to the first nucleotide sequence. The quadruplex-forming sequence of the first nucleotide sequence includes the formula G₃₊N₁₋₇ G₃₊N₁₋₇G₃₊X₁₋₇G₀₋₃, where G represents guanine, N represents any nucleotide (C, G, T, or A), and X represents any nucleotide (C, G, T, or A) or a detectable labeling compound. The G content of the quadruplex-forming sequence can be equal to or greater than 70%. In some examples, the quadruplex-forming sequence comprises GGGCGGGCGGG(2AP)GGG (SEQ ID NO: 1). In some examples, the quadruplex-forming sequence comprises at least 70% sequence identity to SEQ ID NO: 1.

The second nucleotide sequence comprises a stem sequence and a toehold sequence operably linked to the stem sequence. The toehold sequence is sufficiently complementary to the target nucleic acid to hybridize with it. The stem sequence is also sufficiently complementary to a portion of the target nucleic acid to hybridize with it. The stem sequence is furthermore sufficiently complementary to a portion of the quadruplex-forming sequence to hybridize with it. In some aspects, the stem sequence comprises the formula C₃₊N₁₋₇ (where N is any nucleotide). In some examples, the stem sequence comprises CCCGC (SEQ ID NO: 2). In some examples, the stem sequence comprises at least 70% sequence identity to SEQ ID NO: 2.

In some examples, the oligonucleotide takes a hairpin conformation under a first set of conditions, and wherein the first nucleotide sequence of the oligonucleotide takes a quadruplex-conformation under a second set of conditions. The first set of conditions, for example, can include less potassium in the surrounding environment than the second set of conditions. In another example, the first set of conditions can include more cesium in the surrounding environment than the second set of conditions.

Methods for detecting a target nucleic acid are also disclosed herein. The methods can be performed without the use of enzymes. The methods include obtaining a sample that includes the target nucleic acid, providing an oligonucleotide as described above, and binding the target sequence of the target nucleic acid to the second nucleotide sequence. Binding the target sequence to the second nucleotide sequence induces the quadruplex-forming sequence to take a quadruplex conformation. The method further includes detecting the oligonucleotide in the quadruplex conformation and associating the detection of the quadruplex-conformation with the binding of the target sequence. The quadruplex conformation can be detected, for example, by registering a change an optically detectable property.

Some example methods of detecting a target nucleic acid further include forming a hairpin conformation with the oligonucleotide prior to binding the target sequence. Forming a hairpin conformation includes hybridizing a stem sequence of the second nucleotide to the quadruplex-forming sequence of the first nucleotide sequence. Some example methods further include stabilizing the hairpin configuration with cesium ions, magnesium ions, or both. Binding the target sequence to the second nucleotide sequence can include first binding the target sequence to a toehold sequence that is operably linked to the stem sequence while the oligonucleotide is in the hairpin conformation. Binding the target sequence to the second nucleotide sequence can further include binding the target sequence to the stem sequence while the quadruplex-forming sequence dissociates from the stem sequence. Some example methods of detecting a target nucleic acid further include forming heating the sample, thereby inducing the oligonucleotide to revert from the quadruplex conformation to the hairpin conformation.

Some example methods of detecting a target nucleic acid further include stabilizing the quadruplex conformation with potassium ions. In some examples, the potassium ion concentration is less than 10 milliMolar. In some example methods, the oligonucleotide is first dissolved in a buffer comprising cesium ions and/or magnesium ions before potassium ions are added.

In some example methods, detecting the oligonucleotide in the quadruplex conformation further comprises registering a change in an optically detectable property. Registering a change in an optically detectable property can include measuring UV absorption, measuring fluorescence, or both. Some example methods further include quantifying the target nucleic acid.

The methods and oligonucleotides for detecting a nucleic acid, described above, can be used to diagnose a subject with a disease or a disorder, to make a prognosis of a disease or disorder, or to predict the development of a future disease or disorder. A bodily sample is obtained from the subject, nucleic acids are extracted from the bodily sample, and the nucleic acids from the bodily sample are contacted with an oligonucleotide. The oligonucleotide includes the first nucleotide sequence having the quadruplex-forming sequence and the second nucleotide sequence having the stem and toehold sequences, as described above. If the target nucleic acid is present within the nucleic acids from the bodily sample, it binds the second nucleotide sequence, inducing the oligonucleotide to take a quadruplex conformation. The quadruplex conformation is detected and correlated with a diagnosis of the disease or disorder, a prognosis of the disease or disorder, or with a possibility that the disease or disorder may develop in the future. The target nucleic acid can further be quantified and compared to a predetermined threshold range or value for the purposes of correlating the quantity of the target nucleic acid with a diagnosis of the disease or disorder, a prognosis of the disease or disorder, or the development of the disease or disorder.

### DESCRIPTION OF DRAWINGS

The device is explained in even greater detail in the following drawings. The drawings are merely exemplary and certain features may be used singularly or in combination with other features. The drawings are not necessarily drawn to scale.

FIGS. 1A-1C show schematic diagrams of the tertiary structure transformation (ST) reaction, wherein the oligonucleotide substrate begins in a hairpin conformation and transforms to the quadruplex conformation. FIG. 1A shows the reaction components. The quadruplex-forming sequence (medium grey) with 2-aminopurine (2AP, light grey) is incorporated within the hairpin with a second, non-quadruplex forming nucleotide sequence (dark grey) left partially available for binding to the target sequence (black). FIG. 1B shows an example oligonucleotide sequence with a secondary structure of the hairpin conformation. FIG. 1C shows the detailed catalytic pathway of the ST reaction.

FIGS. 2A and 2B show schematics of an enthalpy-driven signal amplifier. FIG. 2A shows reaction components, and FIG. 2B shows the pathway. The product duplex is ˜10 bp longer than stem segments of both hairpins. This overcomes the entropic penalty of the complex formation and makes the overall reaction Enthalpy-driven.

FIGS. 3A and 3B show schematics of an entropy-driven signal amplifier. FIG. 3A shows reaction components, and FIG. 3B shows the pathway. The substrate and product duplexes contain the same number of bps. Since substrate duplex is trimolecular while product duplex is bimolecular the overall reaction is entropy-driven.

FIGS. 4A-4D show schematics of a G-tetrad. FIG. 4A shows the sequence and schematic diagram of G3T quadruplex. FIG. 4B shows a three-dimensional (3D) representation of G3T.

FIG. 4C shows the surface of a 3D model of G3T. FIG. 4D shows the 3D model of G3T unwrapped into a two-dimensional map.

FIG. 5 shows a schematic of a quadruplex priming amplification.

FIG. 6 shows UV melting curves measured at 4 micromolar at 260 nm (upper row) and at 295 nm (middle row), and a fluorescence melting curves measured at 2 micromolar (bottom row, RFU=relative fluorescence units) of the ST hairpin confirmation in the presence of different amounts of K⁺ listed above the columns. Solid and dashed lines correspond to heating and cooling curves, accordingly. Vertical lines, respectively, correspond to Tm values of G3N quadruplex (labeled 17) and ST substrate (hairpin, labeled 19).

FIG. 7 shows the fluorescence effects of invading 5′-GGGCGGGCGGG(2AP)GGG-3′ (SEQ ID NO: 1) (2 μM) quadruplex by a complementary strand (2.2 μM).

FIG. 8 shows the background activity of ST hairpin monitored by 2AP fluorescence.

FIG. 9A shows a schematic of exemplary ST hairpin and targets.

FIG. 9B shows single-turnover kinetics in the presence of 2 mM KCl (2 mM KCl, 48 mM CsCl and 2 mM MgCl₂) at different temperatures.

FIG. 10 shows ST kinetics conducted on the ST substrate with target T1, 5′-CTGGAAGCGGG-3′ (SEQ ID NO: 3), at different K+ concentration at 37° C. The black points correspond to the reaction initiated by adding the target into the ST substrate that was prepared by folding the hairpin in Cs+- and Mg2+-containing buffer and adding K+ later. The grey points correspond to the kinetic experiments in the same mixtures restarted by the heat step. The rates are derived from a second-order exponential fit.

FIG. 11 shows fluorescence quantification graphs demonstrating the steady-state ST kinetics of 2 μM hairpin substrate in the presence of different amounts of target (5′-TGGAAGCGGG-3′ (SEQ ID NO: 4)) in 2 mM KCl, 48 mM CsCl, 2 mM MgCl2 (left panel) and 10 mM KCl, 40 mM CsCl, 2 mM MgCl2 (right panel) at 37° C. Concentrations of the target, with corresponding color code, are shown at the right panel and are the same for the left panel.

### DETAILED DESCRIPTION

The following description of certain examples of the inventive concepts should not be used to limit the scope of the claims. Other examples, features, aspects, embodiments, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing aspects. The invention extends to any novel one, or any novel combination, of the features disclosed in this specification (including any accompanying claims, abstract, and drawings), or to any novel one, or any novel combination, of the steps of any method or process so disclosed.

Terms used throughout this application are to be construed with ordinary and typical meaning to those of ordinary skill in the art. However, Applicant desires that the following terms be given the particular definition as defined below.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

“Optional” or “optionally” means that the subsequently described event or circumstance may or may not occur, and that the description includes instances where said event or circumstance occurs and instances where it does not.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Aspects defined by each of these transition terms are within the scope of this invention.

The term “subject” refers to any individual who seeks a diagnosis, a prognosis, or receives a treatment. The subject can be a vertebrate, for example, a mammal. Thus, the subject can be a human or veterinary patient. The term “patient” refers to a subject under the treatment of a clinician, e.g., physician.

The term “sample from a subject” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The terms “prevent,” “preventing,” “prevention,” and grammatical variations thereof as used herein, refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length. The nucleic acid can include any deoxyribonucleic acid (DNA) or ribonucleic acid (RNA), DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “oligonucleotide” refers to a polymeric form of nucleotides of any length, either deoxyribonucleotides or ribonucleotides, or analogs thereof. Oligonucleotides may have any three-dimensional structure, and may perform any function, known or unknown. Oligonucleotide can refer to a sequence of genomic, synthetic, or recombinant origin and may be double-stranded or single-stranded, whether representing the sense or anti-sense strand. The following are non-limiting examples of oligonucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, genomic DNA, synthetic DNA, nucleic acid probes, and primers. The oligonucleotide may contain chemical modifications. An oligonucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. An oligonucleotide may be further modified after polymerization, such as by conjugation with a labeling component. The term also refers to both double- and single-stranded molecules.

An oligonucleotide is composed of a specific sequence of four nucleotide bases: adenine (A); cytosine (C); guanine (G); thymine (T); and uracil (U) for thymine (T) when the oligonucleotide is RNA. Thus, the term “oligonucleotide sequence” is the alphabetical representation of an oligonucleotide molecule. This alphabetical representation can be input into databases in a computer having a central processing unit and used for bioinformatics applications such as functional genomics and homology searching.

The term “detectable label,” or “labeling compound” refers to any moiety that can be selectively detected in a screening assay. Examples include without limitation, radiolabels, (e.g., .sup.3H, .sup.14C, .sup.35S, .sup.125I, .sup.131I), affinity tags (e.g. biotin/avidin or streptavidin, binding sites for antibodies, metal binding domains, epitope tags, FLASH binding domains—See U.S. Pat. Nos. 6,451,569; 6,054,271; 6,008,378 and 5,932,474—glutathione or maltose binding domains) fluorescent or luminescent moieties (e.g. fluorescein and derivatives, GFP, rhodamine and derivatives, lanthanides etc.), fluorescent nucleobases (e.g., 2-aminopurine), and enzymatic moieties (e.g. horseradish peroxidase, .beta.-galactosidase, .beta.-lactamase, luciferase, alkaline phosphatase). Such detectable labels can be formed in situ, for example, through use of an unlabeled primary antibody which can be detected by a secondary antibody having an attached detectable label.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

By “probe,” “primer,” is meant a DNA or RNA molecule of defined sequence that can base-pair to a second, complementary DNA or RNA sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions.

By “hybridizes” is meant that an oligonucleotide recognizes and physically interacts (that is, base-pairs) with a substantially complementary nucleic acid under high stringency conditions, and does not substantially base pair with other nucleic acids. The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art.

When hybridization occurs in an antiparallel configuration between two single-stranded oligonucleotides, those oligonucleotides are described as “complementary”. “Complementarity” is quantifiable in terms of the proportion of bases in opposing strands that are expected to form hydrogen bonding with each other, according to generally accepted base-pairing rules.

The term “identity” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. Neither N- nor C-terminal extensions nor insertions shall be construed as reducing identity or homology. An oligonucleotide or oligonucleotide region that has a certain percentage (for example, 80%, 85%, 90%, or 95%) of “sequence identity” to another sequence means that, when aligned, that percentage of bases are the same in comparing the two sequences. This alignment and the percent homology or sequence identity can be determined using software programs known in the art. In one aspect, default parameters are used for alignment. In one aspect a BLAST program is used with default parameters. In one aspect, BLAST programs BLASTN and BLASTP are used with the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank, CDS translations+SwissProtein+SPupdate+PIR.

Disclosed herein are oligonucleotides and methods for detecting a target nucleic acid. The oligonucleotides include a quadruplex-forming sequence. Prior to binding the target nucleic acid, the quadruplex-forming sequence is in a non-quadruplex conformation. Upon binding the target nucleic acid, the sequence transforms to the quadruplex conformation, which can be detected, for example, using optical methods. The methods are based on thermodynamic properties and do not require the use of enzymes. The methods and oligonucleotides can be used to diagnose a subject with a disease or a disorder, prognose the outcome of a disease or disorder, or predict the development of a future disease or disorder.

Due to their base pairing properties, nucleic acid sequences can often form specific structures under certain solution conditions. For example, in the presence of certain metal ions (e.g., K⁺), short guanine (G)-rich sequences fold into a structure known as a G-quartet or quadruplex. Quadruplexes are high-ordered DNA and RNA structures formed from G-rich sequences that are built around tetrads of hydrogen bonded guanine bases. The synthetic polynucleotides poly(dG) and poly(G) were determined to form four-stranded helical structures, with the G-tetrads stacked on one another, analogous to Watson-Crick base pairs in duplex DNA.

Quadruplexes are very stable and biophysical studies have shown that they possess natural optical properties (e.g., absorb light at 300 nm) that distinguish them from other secondary structures. When G-rich sequences with the potential to form a quadruplex are incorporated into DNA substrates they are initially in the quenched state. Upon enzymatic activity (i.e., strand cleavage or strand-exchange) the released sequence folds into a quadruplex and becomes visible when monitored by absorption or fluorescence spectroscopy.

Quadruplexes can be formed from one, two, or four separate strands of DNA (or RNA) and can display a wide variety of topologies, which are in part a consequence of the various possible combinations of strand directions, as well as variations in loop size and sequence. They can be defined in general terms as structures formed by a core of at least two stacked G-tetrads, which are held together by loops arising from the intervening mixed sequence nucleotides that are not usually involved in the tetrads themselves. The combination of the number of stacked G-tetrads, the polarity of the strands, and the location and length of the loops would be expected to lead to a plurality of G quadruplex structures, as is found experimentally (12).

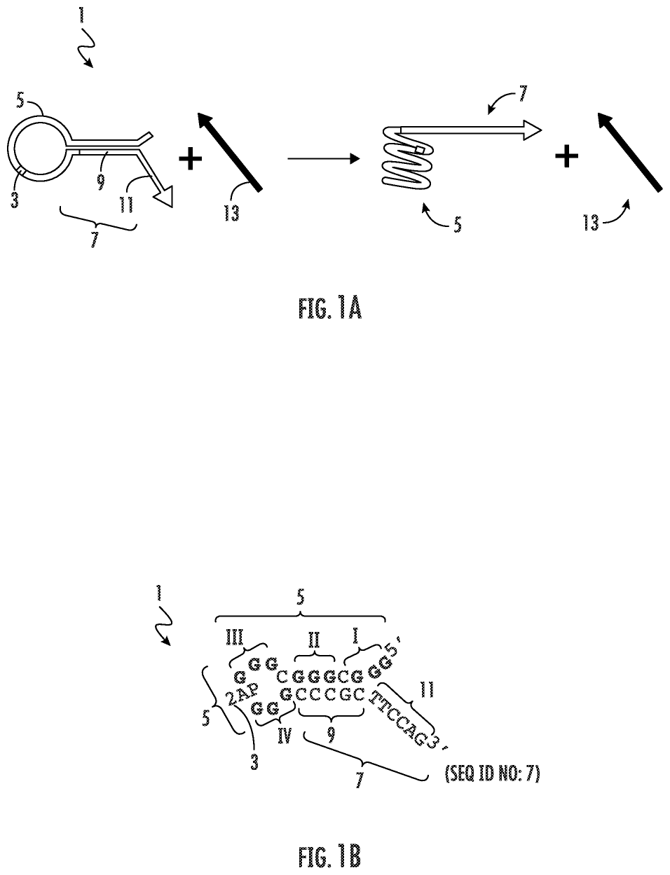

The oligonucleotides disclosed herein can be used to detect a target nucleic acid in a non-enzymatic nucleic acid detection assay. Referring to FIG. 1A, the oligonucleotide 1 includes a first nucleotide sequence that includes a quadruplex-forming sequence 5, and a second nucleotide sequence 7 that is operably linked to the first nucleotide sequence. The quadruplex-forming sequence 5 of the first nucleotide sequence comprises the formula G₃₊N₁₋₇G₃₊N₁₋₇G₃₊X₁₋₇G₀₋₃. The guanine (G) content of the quadruplex-forming sequence is equal to or greater than 70% (including greater than 75%, greater than 80% and greater than 85%, for example). N represents any nucleotide (C, G, T, or A) and X can represent any nucleotide (C, G, T, or A), or a detectable labeling compound, such as, for example 2-aminopurine (2AP). In some aspects, the quadruplex-forming sequence comprises GGGCGGGCGGG(2AP)GGG (SEQ ID NO: 1). In some aspects, the quadruplex-forming sequence has at least 70% sequence identity to SEQ ID NO: 1 (including at least 70% sequence identity, at least 75% sequence identity, at least 80% sequence identity, at least 85% sequence identity, at least 90% sequence identity, and at least 95% sequence identity to SEQ ID NO: 1).

The second nucleotide sequence 7 of the oligonucleotides disclosed herein includes a stem sequence 9 and a toehold sequence 11. As shown in FIGS. 1A-1C, the stem sequence 9 of second nucleotide sequence 7 is sufficiently complementary to a portion of the quadruplex-forming sequence 5 to hybridize with it in a hairpin conformation. The stem sequence 9 is also sufficiently complementary to a portion of the target nucleic acid 13 to hybridize with it. The toehold sequence 11, which is operably attached to the stem sequence 9, is sufficiently complementary to a portion of the target nucleic acid 13 to hybridize with it. The stem sequence 9 is hybridized to the quadruplex-forming sequence 5 prior to the reaction with the target sequence 13, when the oligonucleotide 1 is in a hairpin conformation.

The hairpin conformation can be stabilized by the use of a buffer comprising cesium ions, magnesium ions, or a combination thereof. Altering the concentrations of the ions in the buffer affects the stability of the hairpin structure. In some examples, the hairpin structure is stabilized by a buffer comprising less than 100 milliMolar Mg²⁺, including, for example, less than 70 milliMolar Mg²⁺, less than 50 milliMolar Mg²⁺, less than 30 milliMolar Mg²⁺, less than 10 milliMolar Mg²⁺, less than 9 milliMolar Mg²⁺, less than 8 milliMolar Mg²⁺, less than 7 milliMolar Mg²⁺, less than 6 milliMolar Mg²⁺, less than 5 milliMolar Mg²⁺, less than 4 milliMolar Mg²⁺, less than 3 milliMolar Mg²⁺, less than 2 milliMolar Mg²⁺, and less than 1 milliMolar Mg²⁺. In some examples, the hairpin structure is stabilized by a buffer comprising less than 200 milliMolar Cs⁺, including, for example, less than 150 milliMolar Cs⁺, less than 100 milliMolar Cs⁺, less than 80 milliMolar Cs⁺, less than 70 milliMolar Cs⁺, less than 60 milliMolar Cs⁺, less than 50 milliMolar Cs⁺, less than 40 milliMolar Cs⁺, less than 20 milliMolar Cs⁺, less than 10 milliMolar Cs⁺, less than 5 milliMolar Cs⁺, and less than 1 milliMolar Cs⁺.

Upon binding of target sequence 13 to toehold sequence 11 (FIG. 1C, step i), quadruplex forming sequence dissociates from stem sequence 9 (FIG. 1C, step ii and step iii), allowing the binding of the rest of the target sequence 13 to the stem sequence 9. In some aspects, the stem sequence 9 includes the formula C₃₊N₁₋₇. For example, the stem sequence 9 can include CCCGC (SEQ ID NO: 2), as shown in FIG. 1B. In some aspects, the stem sequence has at least 70% sequence identity to SEQ ID NO: 2 (including at least 70% sequence identity, at least 75% sequence identity, at least 80% sequence identity, at least 85% sequence identity, at least 90% sequence identity, and at least 95% sequence identity to SEQ ID NO: 2).

The oligonucleotide 1 takes a hairpin conformation under a first set of conditions. Under a second set of conditions, the quadruplex-forming sequence 5 of the first nucleotide sequence takes a quadruplex-conformation 15 (FIG. 1C, step iv). The target sequence 13 dissociates from the oligonucleotide 1 when the oligonucleotide 1 takes the quadruplex conformation 15 (FIG. 1C, step v). The materials in the surrounding environment (i.e., buffer) can cause the quadruplex conformation to be more or less favorable. In an example, the first set of conditions might include less potassium in the environment than the second set of conditions (i.e., increasing potassium favors the quadruplex conformation). In another example, the first set of conditions might include more cesium in the environment than the second set of conditions (i.e., increasing cesium favors the hairpin conformation). In an example, the first set of conditions might include no target sequence in the environment, whereas the second set of conditions does include target sequence in the environment (i.e., target sequence drives a change from the hairpin conformation to the quadruplex conformation). In an example, the first set of conditions might include less target sequence in the environment than the second set of conditions.

Further, a quadruplex-forming nucleotide sequence may have a detectable label incorporated therein for detection purposes. Such a label may be chosen from labels that are known to those of ordinary skill in the art. Such labels include, but are not limited to, fluorescent labels. In one particular aspect, the primer may have a fluorescent label incorporated therein. And in a particular aspect, such a label may include 2AP. A unique property of the amplification process disclosed herein is that 2AP incorporated in the oligonucleotide results in strong fluorescence emission upon formation of a structure such as a quadruplex, thus serving as a sensitive detection probe for binding of the target sequence and subsequent formation of the quadruplex.

Other fluorescent nucleotides that can be utilized for detection of the quadruplex conformation can include pteridine analogs: 3-methyl isoxanthopterin (3MI) (Ex348, Em431), 6-methylisoxanthopterin (6MI) (Ex340, Em430) and (4-amino-6-methyl-8-(2¢-deoxy-â-D-ribofuranosyl)-7(8H)-pteridone (6AMP) (Ex330, Em435). As mentioned above, certain optical changes are naturally associated with the transformation to the quadruplex conformation. As such, it is also possible that the tertiary substrate transformation (from hairpin to quadruplex) can be detected without the use of added fluorophores or labeling compounds.

Methods of detecting a target nucleic acid are disclosed herein. The methods include obtaining a sample that includes the target nucleic acid. As described above, an oligonucleotide is provided that includes a first nucleotide sequence comprising a quadruplex-forming sequence and a second nucleotide sequence operably linked to the first nucleotide sequence, the second nucleotide sequence being sufficiently complementary to the target sequence to hybridize therewith. In some aspects, the oligonucleotide forms a hairpin conformation by hybridizing a stem sequence of the second nucleotide to the quadruplex-forming sequence of the first nucleotide sequence. The methods further include binding the target sequence to the second nucleotide sequence. Binding the target sequence to the second nucleotide sequence can include first binding the target sequence to a toehold sequence that is operably linked to the stem sequence while the oligonucleotide is in the hairpin conformation, then binding the target sequence to the stem sequence while the quadruplex-forming sequence dissociates from the stem sequence. Dissociation of the quadruplex-forming sequence from the stem sequence induces the quadruplex-forming sequence to take a quadruplex conformation, which can be detected, for example, by changes in optically detectable properties. Thus, the presence of the target sequence induces a detectable confirmation change in the oligonucleotide. Detection of the confirmation change therefore confirms the presence of the target sequence in the original sample. The oligonucleotide can be induced to revert from the quadruplex conformation back to the hairpin conformation by heating the sample.

In some aspects of the method, the quadruplex conformation is stabilized using potassium ions. The potassium concentration can be from 0.1 milliMolar to 100 milliMolar or more. The potassium concentration of the assay can be optimized depending on the desired results. For example, higher concentrations of potassium will yield faster reaction rates, but may lead to greater leakage than lower potassium concentrations. In one aspect, leakage can be reduced or eliminated by keeping the potassium concentration at less than 10 milliMolar (for example, at less than 1 milliMolar, less than 2 milliMolar, less than 3 milliMolar, less than 4 milliMolar, less than 5 milliMolar, less than 6 milliMolar, less than 7 milliMolar, less than 8 milliMolar, less than 9 milliMolar, and less than 10 milliMolar). However, advantages such as faster reaction rates may make concentrations of 10 milliMolar or greater desirable in certain aspects (for example, 10 milliMolar or greater, 20 milliMolar or greater, 30 milliMolar or greater, 40 milliMolar or greater, 50 milliMolar or greater, 60 milliMolar or greater, 70 milliMolar or greater, 80 milliMolar or greater, 90 milliMolar or greater, and 100 milliMolar or greater). In some aspect, the substrate is first dissolved in buffer comprising cesium and/or magnesium ions prior to the addition of the potassium ions.

In some aspects, the oligonucleotide in the quadruplex conformation is detected by registering a change in an optically detectable property. For example, changes in UV absorption or fluorescence emission can be measured and correlated to the presence of the quadruplex conformation. Specifically, upon binding of the target molecules to the hairpin, the quadruplex-forming sequence containing 2AP is released. The 2AP sequence is initially quenched by neighboring guanines in the hairpin. However, once the quadruplex forms, 2AP emits light and the fluorescence can be used to detect, and even quantify, the initial target molecules.

Interestingly, this method can be performed without the enzymes conventionally required for detecting and amplifying a nucleic acid signal. The ability to perform the assay without enzymes reduces cost and enables point of care diagnostics, which can be useful in many scenarios, one of which is in-field diagnostics, or diagnosis of subjects in locations where highly technical medical machinery is unavailable. In addition, non-enzymatic assays have greater stability and a longer shelf life at room temperature than enzymatic assays because they are based on oligonucleotides, which are more stable than enzymes.

The methods and oligonucleotides discussed above can be used to diagnose a subject with a disease or disorder. The methods include obtaining a bodily sample from the subject, extracting nucleic acids from the bodily sample, and contacting the nucleic acids from the bodily sample with the oligonucleotides discussed above. If the target nucleic acid is present within the nucleic acids from the bodily sample, they will bind to the second nucleotide sequence of the oligonucleotide, thereby inducing the oligonucleotide to take a quadruplex conformation. The quadruplex conformation can then be correlated with a diagnosis of the disease or disorder, a prognosis of the disease or disorder, or the development of the disease or disorder. The target nucleic acid sequence might be derived from any pathogen that causes the disease or disorder, including, but not limited to, malaria or tuberculosis. Alternatively, the target nucleic acid might be derived from the subject, and the methods can be used to detect genetic abnormalities or mutations that can lead to a disease or disorder. Likewise, the methods can be used to make a prognosis of a known disease or disorder, or to predict the development of a future disease or disorder.

### Example 1

### Introduction

Due to remarkable self-assembly properties, nucleic acids have been established as a favorable material for both static and dynamic nanotechnologies (1, 2). One of the main components of dynamic nanotechnologies is the driving force that allows unidirectional operations in an isolated system (i.e., irreversible transform of substrates into products or transport of an object from point A to point B without external stimuli) (3-6). Depending on the energy requirement, the DNA nanomachines can be divided into two major categories: technologies that employ enzymes (i.e., Ribozymes, DNAzymes or proteinous enzymes) to modify covalent bonds; and non-enzymatic technologies in which no covalent bonds are modified (1). The enzymatic technologies usually are based on cleavage of intermediate complexes. This allows spontaneous dissociation of the substrate and next round of activity in a steady-state, linear manner. Since in non-enzymatic assays no covalent bonds are modified, product can be used in the various downstream applications, i.e., cascading (6), or can serve as autocatalyst to accelerate reactions (4). Therefore, the non-enzymatic technologies are more versatile and induce strong interest due to their practical applications (i.e., point-of-care diagnostics) (7).

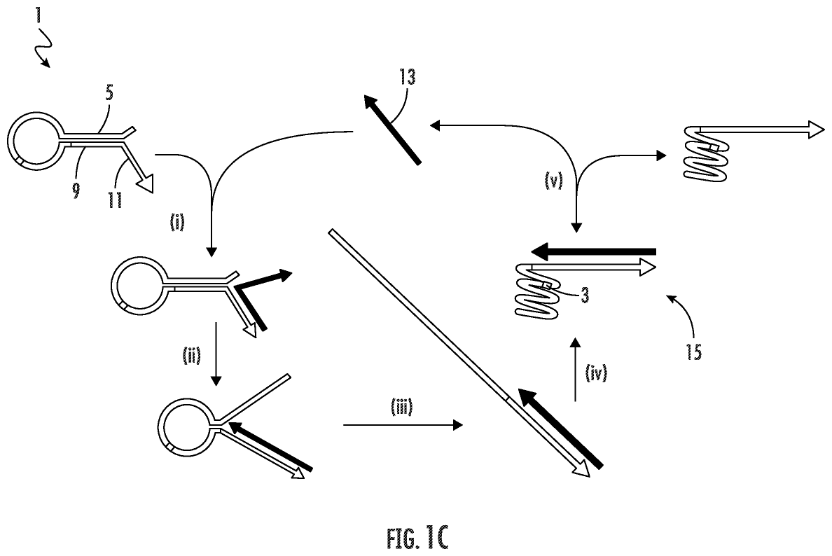

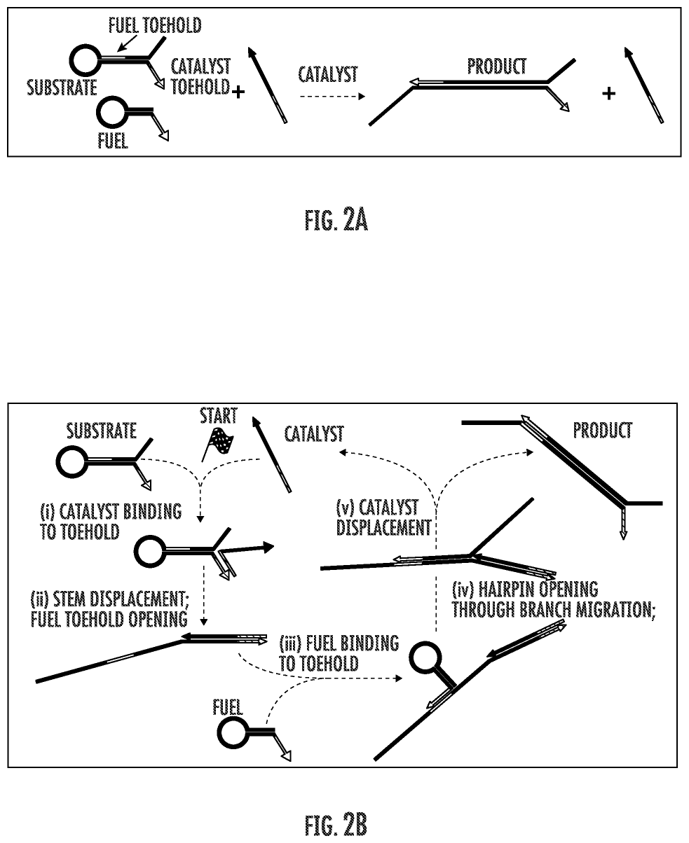

Dynamic nanotechnologies (i.e., gates, signal amplifiers, locomotives) are based on rearrangements between DNA duplexes. To make the reactions unidirectional, duplexed substrates are designed to be thermodynamically less favorable than their respective product duplexes (4, 6). As a result, the reactions are thermodynamically (enthalpically or entropically based on a system) driven, but demonstrate detrimental background-activity, or leakage (observed product formation before the addition of catalyst). To better understand the mechanism and challenges of reactions based on DNA-to-DNA transitions, consider an enthalpy-driven reaction. Reaction components for an enthalpy-driven reaction are shown in FIG. 2A, and the pathway is shown in FIG. 2B (6). In this reaction, two hairpin molecules (substrate and fuel) are capable of forming one bi-molecular product duplex. The product duplex is ˜10 bp longer than stem segments of both hairpins. This overcomes the entropic penalty of the complex formation and makes the overall reaction thermodynamically (enthalpically in this case) favorable. It is assumed that until catalysis, the substrate and fuel hairpins would not react with each other because no complementary segments are exposed (fuel toehold, light grey, is constrained in the substrate hairpin, FIG. 2A). Thus, the formation of a more favorable final product is kinetically trapped in the metastable substrate. This condition is achieved by annealing the substrate and fuel hairpins separately and mixing together just before the reaction. In other words, by combining the individually thermodynamically stable molecules, the reaction receives a driving energy and is ready for signal amplification. The reaction is then triggered by addition of the catalyst that binds to the exposed toehold (dark grey) of the substrate hairpin (step i) and unfolds it through branch migration (step ii). This exposes the fuel toehold (light grey segment in substrate) and allows the fuel hairpin to bind to the substrate (step iii). After the second branch migration (step iv) the catalyst is released (step v). At this stage, the liberated catalyst is available to bind another substrate molecule and initiate subsequent strand-displacement cycles. The entropically driven reactions are essentially the same with only difference that a tri-molecular substrate and a fuel strand are capable of forming a bi-molecular base-paired product, which is entropically more favorable than the initial tri-molecular substrate (see FIG. 3A and FIG. 3B).

In conclusion, both enthalpically and entropically driven reactions are governed by the same toehold activation/inactivation cycles. Specifically, before triggering the reactions, the catalyst toehold (dark grey) is exposed and active, while the fuel toehold (light grey) is inactivated by constraining into the substrates. The first strand-exchange event is accompanied by activation of the fuel toehold and simultaneous inactivation of the catalyst toehold. The second, and final, strand-exchange event constrains the fuel strand but exposes catalyst toehold in the product molecules, which can initiate undesired reverse reaction. However, the catalyst switches to the next substrate due to thermodynamic favorability of the product over the substrate (or incapability to invade the product). So, at the end of the cycle, the driving force of the reaction inactivates the exposed toehold and blocks the reverse reaction. In other words, the main role of the driving force is to ensure unidirectional nature of the reaction by inactivating the exposed toehold of the product.

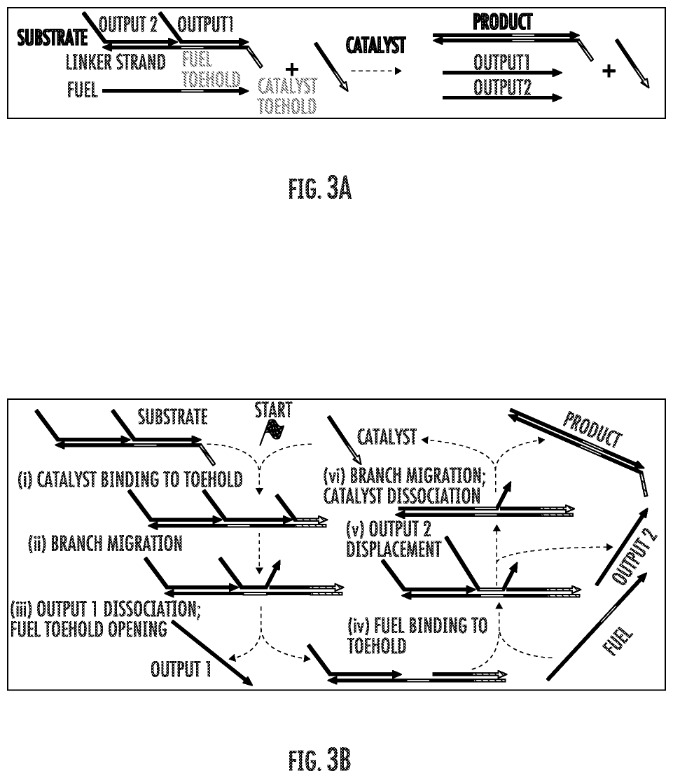

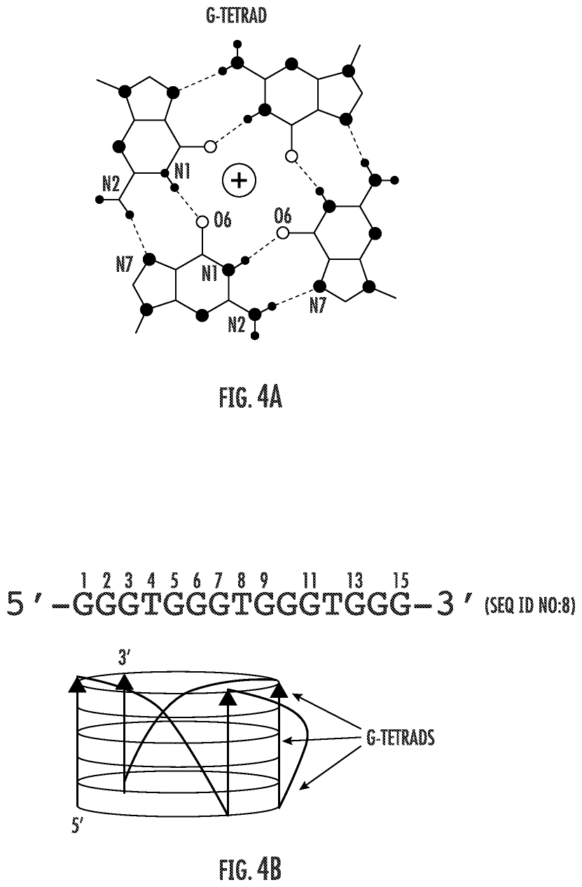

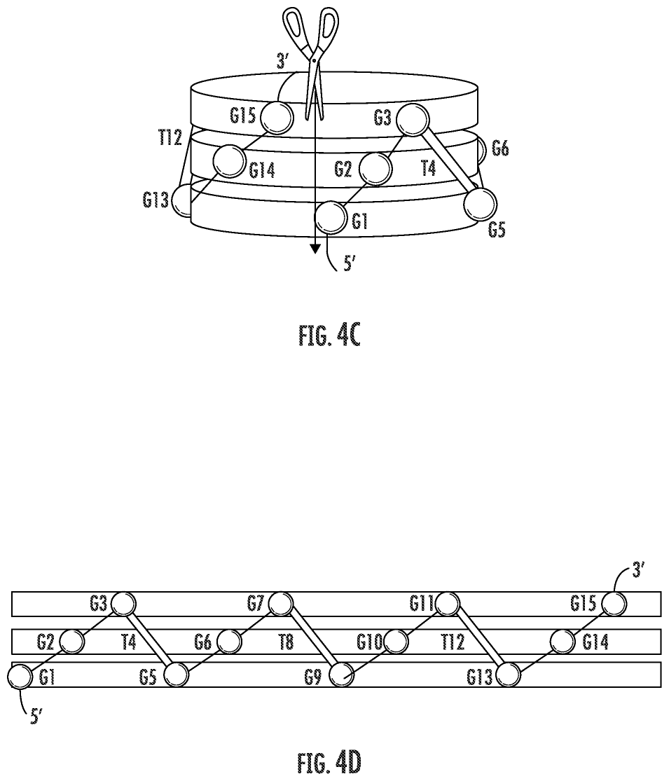

Can the reverse reactions be blocked differently? For instance, by designing a reaction based on structural transformation (ST) between a DNA duplex and some highly packed non-canonical structure. This approach has already been used to transform thermocycling PCR (Polymerase Chain Reaction) into isothermal QPA (Quadruplex Priming Amplification) (8). The assay is based on GGGNGGGNGGGNGGG (SEQ ID NO: 5) (G3N) sequence, where N=G, A, C or T. G3N forms highly structured and unusually stable quadruplex. FIGS. 4A-4D show schematics of a G3T G-tetrad (wherein N=T for the G3N sequence), GGGTGGGTGGGTGGG (SEQ ID NO: 8). Twelve out of fifteen nucleotides are involved in quadruplex formation and only three nucleotides, forming propeller loops, are exposed. As a result, (i) invasion of the quadruplex by a complementary strand is hindered even under experimental conditions favorable for the duplex formation (single-nt loops are incapable to play role of toeholds and initiate the quadruplex invasion) (see Results and Discussions) (9, 10), and (ii) G3N quadruplex alone can reach thermal stability higher than corresponding duplex (8, 11). QPA takes advantage of these properties and employs truncated (missing the 5′-end guanine) version of G3N as a primer. The missing residue is critical for quadruplex formation. As a result, the truncated sequence anneals/primes to the template without complication (step i, FIG. 5). When polymerase attaches the missing guanine (step ii), the extended primer spontaneously dissociates from the template, folds into a DNA quadruplex (step iii) and the template is ready for the next priming without a thermocycler. So, QPA can be considered as an enzymatic, or polymerase-mediated, ST.

This Example describes a non-enzymatically conducted ST reaction between a monomolecular DNA substrate and a quadruplex product (FIGS. 1A-1C). The G3N sequence (quadruplex-forming sequence 5) is incorporated into a hairpin molecule under buffer conditions favorable for the hairpin formation at specific ratios of Cs+ and K+ ions. After the toehold-mediated strand-displacement, initiated by the active toehold 11, the liberated quadruplex-forming sequence 5 folds into a quadruplex and stays folded after catalyst dissociation (step v, FIG. 1C). The Example analyses the thermodynamic principles of ST reactions and demonstrates that the unidirectional non-enzymatic reaction can be run without thermodynamic favorability by transforming thermodynamically stable substrates into metastable products. This allows to (i) run nanodevices without detrimental background-activity; and (ii) charge the product molecules with a potential energy, which could be used in downstream endergonic activities (i.e., restarting the reaction by a heating step).

As noted above, the approach is based on tertiary structure transformation (ST) between a DNA duplex (hairpin) and a quadruplex (FIG. 1A). The ST-assay employs an oligonucleotide 1 initially in a hairpin confirmation. The oligonucleotide 1 includes a detection mechanism 3, and two major nucleotide sequences. The first nucleotide sequence includes the 15-nt quadruplex-forming sequence 5 (medium grey) with detection mechanism 3 (light grey) for signal detection. The second nucleotide sequence 7 (dark grey) is the portion that does not form a quadruplex. The second nucleotide sequence 7 is a 11-nt complement of the target sequence 13 (black), and it includes a stem sequence 9 and a toehold sequence 11. Initially, in the hairpin conformation, the 5-nt stem sequence 9 of the second nucleotide sequence 7 binds quadruplex-forming sequence 5, and the remaining 6-nt toehold sequence 11 of the second nucleotide sequence 7 hangs off the hairpin to create the toehold for the target sequence 13.

FIG. 1B shows an exemplary full sequence of oligonucleotide 1, 5′-GGGCGGGCGGG(2AP)GGGCCCGCTTCCAG-3′ (SEQ ID NO: 7), folded in the hairpin conformation. The quadruplex-forming sequence 5, abbreviated to G3N, is 5′-GGGCGGGCGGG(2AP)GGG-3′ (SEQ ID NO. 1). The 2-aminopurine, 2AP, is the detection mechanism 3. The guanine-rich sequences of quadruplex-forming sequence 5, which cause the folding into the quadruplex conformation, are labeled I, II, III, and IV. The stem sequence 9, CCCGC (SEQ ID NO: 2), of the second nucleotide sequence 7, is bound to the quadruplex-forming sequence 5, and toehold sequence 11, TTCCAG (SEQ ID NO: 11), hangs off such that it is available for binding to the target sequence 13.

In an example of the ST reaction, shown in FIG. 1C, the target sequence 13 initially hybridizes to the 6-nt toehold sequence 11 (step i) and continues strand-displacement hybridization through branch migration (step ii). At this point the quadruplex is trapped with a single GC base-pair. After unfolding the base-pair (step iii), the quadruplex conformation 15 forms and emits light from detection mechanism 3 (step iv), and target sequence is ready to dissociate and perform next cycle of ST (step (v)).

### Materials and Methods:

DNA oligonucleotides were obtained from Integrated DNA Technologies (Coralville, Iowa). The quadruplex-forming sequence 5, shown in FIG. 1B, is 5′-GGGCGGGCGGG(2AP)GGG-3′ (SEQ ID NO. 1). The target sequence 13 is 5′-CTGGAAGCGGG-3′ (SEQ ID NO: 3). The concentration of the DNA oligonucleotides was determined by measuring UV absorption at 260 nm as described earlier (12). All measurements were performed in 10 mM Tris-HCl, pH 8.7 with the ionic strength adjusted by addition of appropriate salts as indicated in the figure legends.

UV absorption was recorded at 260 and 295 nm as a function of temperature using a Varian UV-visible spectrophotometer (Cary 100 Bio). Fluorescence measurements of 2AP (excitation 310 nm, emission 370 nm) were performed using a Varian spectrophotometer (Cary Eclipse). The devices were equipped with thermoelectrically-controlled cuvette holders. In a typical experiment, oligonucleotide stock solutions (usually 100 μM) were mixed into the desired buffers in optical cuvettes. The oligonucleotide solutions (4 μM in UV and 2 μM in fluorescence experiments) were incubated at 95° C. for a few minutes and annealed at room temperature for 2-3 minutes prior to ramping to the desired starting temperatures. In the case of the ST substrate (hairpin), to ensure that the quadruplex-forming sequence incorporated within the hairpin, the solutions were first annealed in CsCl- and MgCl₂-containing buffers, and KCl was added after temperature annealing and ramping. The melting curves allowed an estimate of melting temperature, Tₘ, the midpoint temperature of the unfolding process. To be sure that ST substrate forms only monomolecular structure, shown in FIG. 1B, concentration dependence of Tm was performed. The measurements revealed that Tm value remains constant over 20-fold increase in strand concentration suggesting formation only monomolecular constructs. Van't Hoff enthalpies, ΔH_{vH}, were also calculated using the following equations: ΔH_{vH}=4 R Tₘ²δα/δT; R is the gas constant and δα/δT is the slope of the normalized optical absorbance or fluorescence versus temperature curve at the Tₘ (13). The Gibbs free energy, at 37° C., was estimated according to the equation ΔG=ΔHᵥₕ(Tₘ−T)/Tₘ.

### Results and Discussion

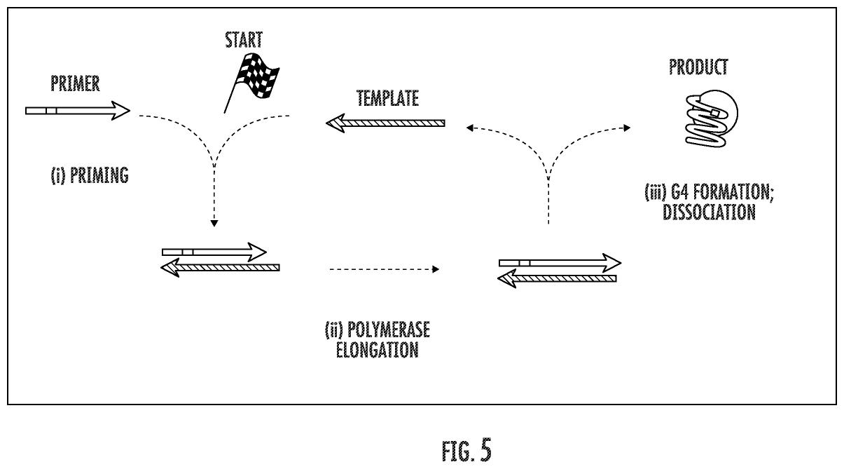

Melting profiles of ST substrate (Tuning ΔG_{Quadr} to ΔG_{Hairpin}): FIG. 6 demonstrates UV and fluorescence unfolding experiments of the ST substrate (FIG. 1B) under different buffer conditions. All buffers contain 50 mM monovalent cations (K⁺ and Cs⁺) and 2 mM Mg²⁺ to keep total ionic strength constant. The K⁺ ions support quadruplex formation, by chelation between G-tetrads. The Cs⁺ and Mg²⁺ ions are incapable of entering the inner core of the structure and are employed to support the hairpin formation. (Note, to make sure that quadruplex-forming G3N sequence is integrated in the stem part of the hairpin, the substrate was dissolved in Cs⁺- and Mg²⁺-containing buffers, annealed and K⁺ was added later). Since the DNA stability depends on the total ionic strength (not the cation type), changing of K⁺ concentration allows tuning of ΔG_{Quadr}, to ΔG_{Hairpin}.

The vertical lines marked 17 in FIG. 6 correspond to Tₘ values of the quadruplex alone, G3N (see Table 1). The vertical lines marked 19 correspond to the actual Tₘ values of the hairpins obtained from the melting curves shown in the panels. Before discussing the data, one should mention that enthalpies of this particular hairpin and the quadruplex are similar (ΔH_{Quadr}=−60 kcal/mol and ΔH_{Hairpin}=−65 kcal/mol). Therefore, Tₘ values closely correlate with ΔG values, and, as a result, the melting profiles reflect overall thermodynamics of the structures. For instance, when red vertical line (Tₘ of the quadruplex) is situated on the right of the blue line (Tₘ of the hairpin) the quadruplex formation is more favorable (ΔG_{Quadr}<ΔG_{Hairpin}) and vice versa.

TABLE 1
Melting temperatures, Tₘ, Van′t Hoff enthalpies,
ΔH_{vH}, and Gibbs free energies at 37° C.,
ΔG, for G3N quadruplex, 5′-GGGCGGGCGGG(2AP)GGG-3′
(SEQ ID NO: 1), and for TST substrate.

| | G3N quadruplex | | TST substrate (hairpin) | |
|---|---|---|---|---|
| | ΔH_{vH} = −58 kcal/mol | | ΔH_{vH} = −65 kcal/mol | |
| [K⁺] (mM)* | Tₘ(° C.) | ΔG (kcal/mol) | Tₘ(° C.) | ΔG (kcal/mol) |
| 0 | — | — | 83.0 | −8.4 |
| 2 | 71.8 | −5.9 | 83.0 | −8.4 |
| 5 | 79.7 | −7.0 | 82.4 | −8.3 |
| 10 | 82.0 | −7.5 | ~80 | −8 |
| 30 | ~90 | −9 | ~68 | −6 |
| 50 | >95 | <−9 | ~65 | −5 |

*The column indicates concentration of K⁺ in the buffers, which contain total 50 mM monovalent cations (K⁺ + Cs⁺) and 2 mM Mg²⁺. For instance, 2 mM [K⁺] corresponds to 2 mM K⁺, 48 mM Cs⁺ and 2 mM Mg²⁺.

OD₂₆₀ is sensitive to duplex formation, while OD₂₉₅ and 2AP-fluorescence monitor the quadruplex formation (8, 14-16). Therefore, in the absence of K⁺ (FIG. 6, far left column), only OD₂₆₀ demonstrates a hyperchromic transition at 83° C. corresponding to equilibrium unfolding of the stem part of the hairpin and well agrees with Tₘ calculated from nearest-neighbor analysis. Both OD₂₉₅ and fluorescence are insensitive to the temperature changes since Cs⁺ and Mg²⁺ doesn't support the quadruplex formation. In the presence of 2 mM and 5 mM K⁺, the ST substrate demonstrates similar melting behaviour because the quadruplex is less stable than the hairpin and, therefore, unlikely to affect melting of the stem. Specifically, in the presence of 2 mM K⁺, Tₘ values of the quadruplex and the hairpin are 72° C. and 83° C., respectively (FIG. 6). Upon the heating cycle, the hairpin starts unfolding around 75° C., which is already above Tₘ of the quadruplex. Therefore, released G3N does not form a quadruplex and the melting demonstrates sigmoidal transition typical for a two-state transition. Upon the cooling cycle, hairpin starts refolding at ˜90° C. and before reaching T_{m, Quadr}, 72° C., G3N-segment is securely incorporated in the stem. Thus, the melting curves are not affected by presence of 2 mM and 5 mM K⁺ ions.

The visible effect of K⁺ ions on the melting curves starts at ≥10 mM K⁺. As expected, the strongest effects are observed at [K⁺]=50 mM (FIG. 6, far right column) with T_{m, Quadr}>95° C. All three heating curves demonstrate cooperative transitions at 65° C.; while the hyperchromic effect in OD₂₆₀ corresponds to unfolding of the stem part of the hairpin, similar effect in OD₂₉₅ corresponds to prompt quadruplex formation that accompanies the hairpin unfolding. The fluorescence curve also demonstrates a cooperative transition (increase in fluorescence) corresponding to the quadruplex formation. The drop in T_{m, Hairpin} (from 83° C., in the absence of K⁺, to 65° C.) is attributed to the quadruplex formation of the released G3N that distorts equilibrium nature of the transition. The cooling curves do not show any transition indicating on incapability of ST product to refold into the hairpin. Similar melting behaviour was observed earlier for bimolecular G3N duplex (8). Qualitatively similar graphs with the following differences are observed at [K⁺]=30 mM: (i) hairpin unfolds at slightly higher temperature, 68° C.; and (ii) OD₂₉₅ and fluorescence measurements demonstrate a reversible transition around 90° C. corresponding to unfolding and refolding of G3N quadruplex. At [K⁺]=10 mM, the quadruplex and the hairpin demonstrate almost the same stabilities and, as a result, only ˜80% of the hairpin molecules are able to refold.

In conclusion, a drastic change in the melting behaviour of the ST substrate takes place around 10 mM K⁺, where both structures, hairpin and quadruplex, melt at similar temperatures, ˜80° C. Below this point, [K⁺]<10 mM, the quadruplex is less favorable than the hairpin (ΔG_{Quadr}>ΔG_{Hairpin}) and has no visible effect on the equilibrium transitions. However, at [K⁺]≥10 mM, the quadruplex becomes more favorable (ΔG_{Quadr}<ΔG_{Hairpin}) and, as a result, the transitions are non-equilibrium and irreversible since the quadruplex can't be incorporated into hairpins upon cooling. Thus, at [K⁺]≥10 mM the ST reactions is thermodynamically favorable (spontaneous), while at [K⁺]<10 mM it is thermodynamically unfavorable.

G3N quadruplex can't be invaded by a complement even under favorable conditions for the invasion: The previous section demonstrates that when formation of G3N quadruplex is more favorable than the hairpin the complementary segment of ST substrate is not able to invade already folded quadruplex. This is in general agreement with earlier observations that, at physiological conditions, invasion of the monomolecular quadruplexes with the complementary strands is unfeasible (17).

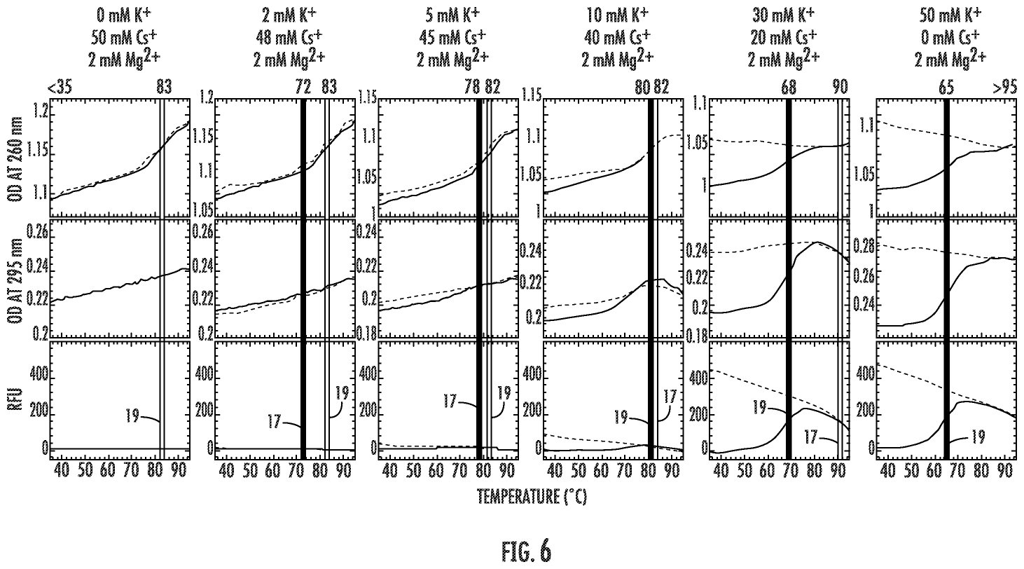

Here, we demonstrate that invasion of G3N quadruplex is problematic even under experimental conditions favorable for the invasion (when final duplex is more favorable than the quadruplex). For this, we studied invasion of 5′-GGGCGGGCGGG(2AP)GGG-3′ (SEQ ID NO: 1) quadruplex with its fully complementary DNA strand, 5′-CCCTCCCGCCCGCCC-3′ (SEQ ID NO: 6). The experiments were conducted in 0.1 mM K⁺ buffer (0.1 mM KCl, 50 mM CsCl and 2 mM MgCl₂). Under this condition G3N quadruplex melts at ˜45° C. with favorable ΔH=−55 kcal/mol, (ΔG=−4 kcal/mol, estimated at 20° C.) while the duplex demonstrates T. of ˜72° C. with ΔH=−120 kcal/mol (ΔG=−18 kcal/mol). Thus, the duplex formation is drastically more favorable than the quadruplex. FIG. 7 demonstrates kinetic measurements, monitored through 2AP-fluorescence, at 20, 30 and 40° C. As expected, before adding the complement, the quadruplex demonstrates strong emission (between 500 and 600 RFU). After adding the complementary strand (at 5 min) one would expect the decrease in fluorescence due to 2AP quenching upon quadruplex invasion (8, 14). This is the case at 30 and 40° C., however at 20° C. no measurable effect is observed (horizontal line between 5 and 22 min, FIG. 7). This clearly indicates that, at 20° C. (where the quadruplex is securely folded), the complement is incapable to invade the quadruplex despite the fact that the final duplex is significantly more favorable. In other words, formation of thermodynamically favorable bimolecular duplex is efficiently restricted by trapping one of the strands in G3N quadruplex. To release G3N sequence from the kinetic trap, the sample was heated to 95° C. and returned into the fluorescence spectrophotometer at experimental temperatures. As expected, after the heating step the sample showed no fluorescence due to full quenching of 2AP upon incorporation in the duplex (8, 14).

Thus, when G3N sequence is securely trapped into the quadruplex (achieved at temperatures ˜20° C. below Tₘ of the quadruplex), the complementary strand is incapable to invade even when the invasion is thermodynamically favorable. Without being wed to theory, this may be attributed to the highly compacted nature of G3N quadruplex employing only single-nt loops that are incapable to serve as toeholds to initiate invasion.

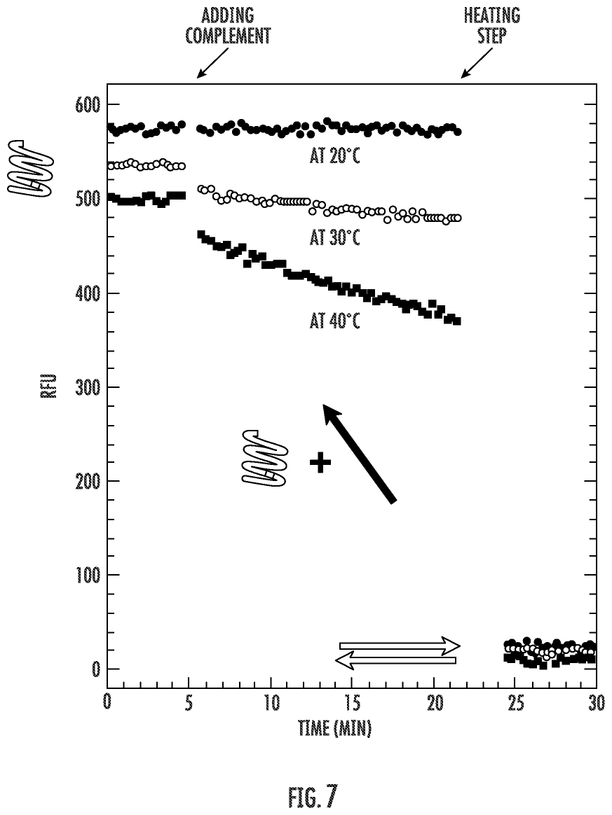

Self ST activity: As shown above, at [K⁺]≥10 mM the product is more favorable than the substrate and, as a result, the ST reaction has a thermodynamic driving force. However, at [K⁺]<10 mM the substrate is more favorable than the product and the reaction doesn't have a driving force. Since the main reason for the leakage for is a thermodynamic driving force, leakage in ST system is expected only at [K⁺]≥10 mM. Indeed, the fluorescence kinetics of ST substrate in the absence of the catalyst/target reveals measurable self-activity at [K⁺]=10 mM and 30 mM above 52° C., while no leakage is observed at [K⁺]=2 mM and 5 mM (FIG. 8). It must be emphasized that at [K⁺]=2 mM and 5 mM G3N quadruplex alone forms highly stable quadruplex (see Table 1, above). The absence of the leakage is likely due to more favorable formation of the hairpin, which overpowers the quadruplex.

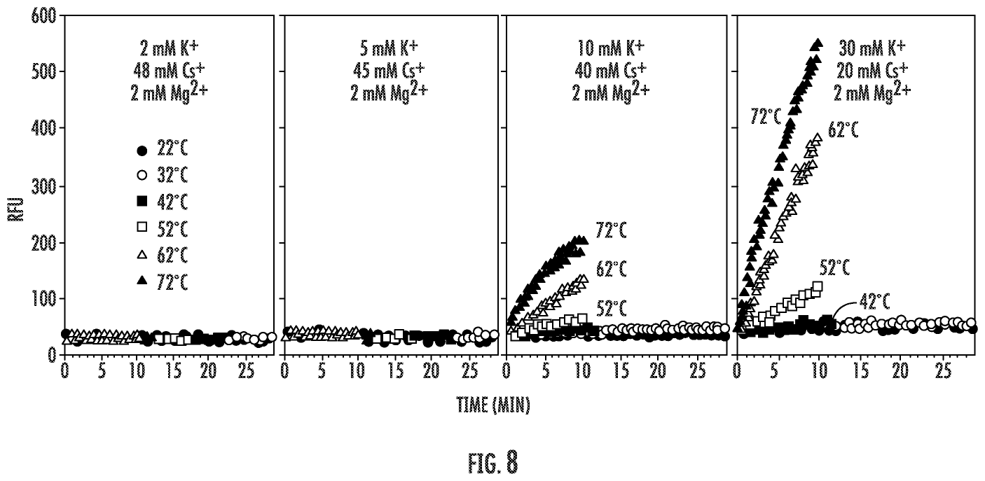

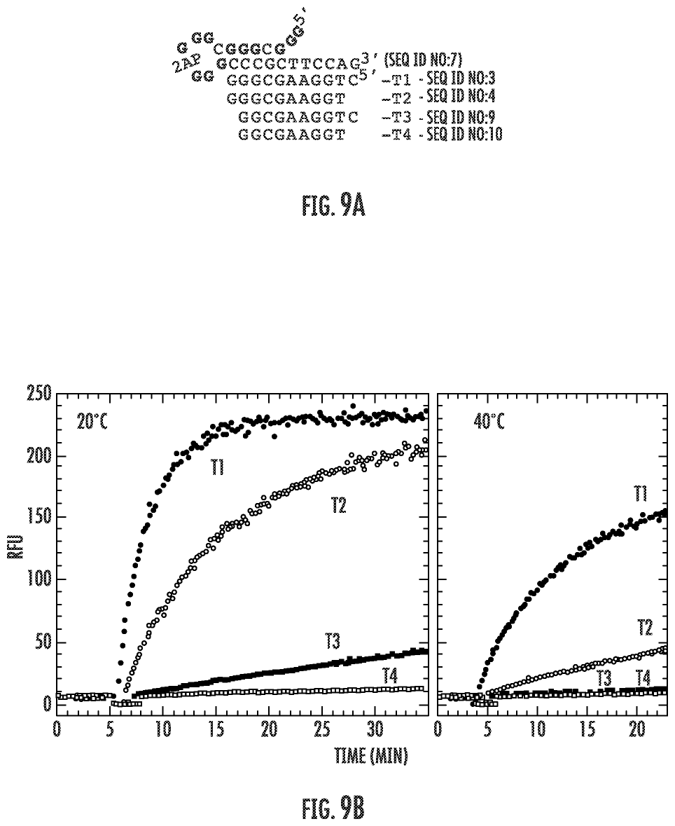

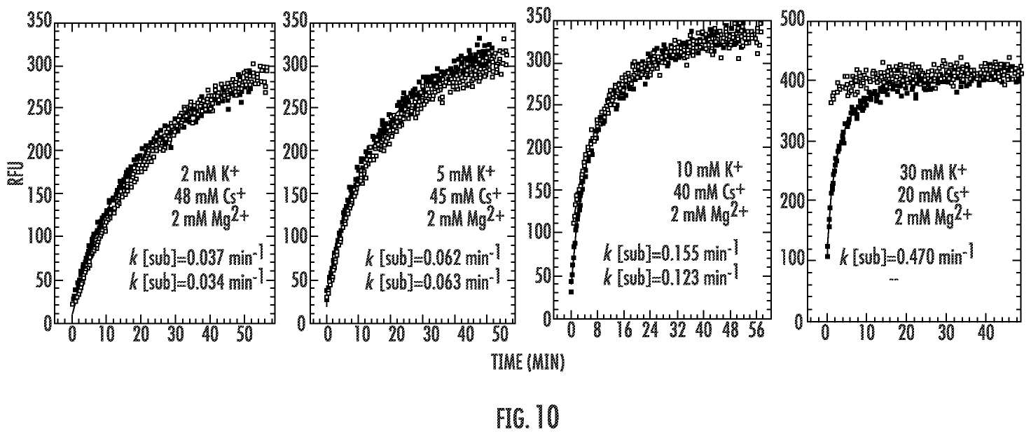

ST activity—Single turnover kinetics: Initially, single turnover reaction was tested using different targets, which demonstrated that 11-nt target, 5′-CTGGAAGCGGG-3′ (SEQ ID NO: 3) (T1) and 10-nt target, 5′-TGGAAGCGGG-3′ (SEQ ID NO: 4) (T2), (5-nt toehold+5-nt invasion) demonstrate fastest kinetics (FIG. 9A and FIG. 9B). FIG. 10 shows kinetics triggered by T1 under different buffer conditions (black points). As expected, reaction accelerates at higher K concentrations. Importantly, thermodynamically unfavorable ST reaction (at [K⁺]<10 mM) transforms stable substrates into thermodynamically less favorable metastable products. This suggest that these reactions can be restarted by heating up the samples (returning the system to thermodynamic equilibrium). Indeed, a heating step (1 min at 90° C.) fully recovers the unfavorable ST kinetics (grey points) (at [K⁺]=2 and 5 mM) and, as expected, has no effect on favorable ST (at [K⁺]=30 mM). This feature can be used to transform product back into substrate without external stimuli and start over the reaction (i.e., reproduce a diagnostic result).

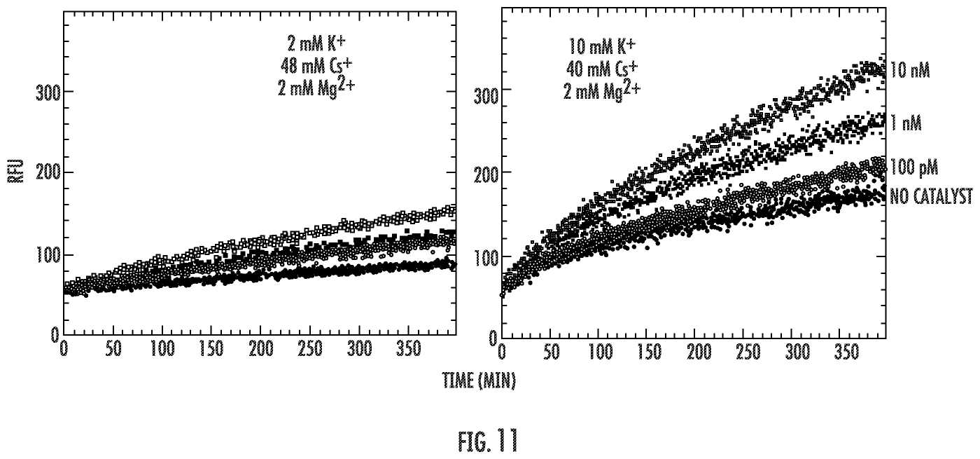

ST activity—Multiple turnover conditions (steady state kinetics): FIG. 11 demonstrates steady-state kinetics of 1 μM substrate in the presence of different concentrations of T2 (0.1-10 nM). The kinetics were tested under experimental conditions corresponding to unfavorable (2 mM K⁺ buffer) and favorable ST (10 mM K⁺ buffer). In 10 mM K⁺ the kinetic curve in the absence of catalyst demonstrates significant leakage due to thermodynamically favorable nature of ST, while in 2 mM K⁺, almost no measurable leakage is observed. For both conditions we observe efficient steady-state activity that allows to differ 100 pM catalyst from the base-line (no target/catalyst).

Unless defined otherwise, all technical and scientific terms used herein have the same meanings as commonly understood by one of skill in the art to which the disclosed invention belongs.

Throughout this application, various publications and patent applications are referenced. The disclosures of these publications in their entireties are hereby incorporated by reference into this application in order to more fully describe the state of the art to which this disclosure pertains. However, it should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Those skilled in the art will recognize, or be able to ascertain using no more than routine experimentation, many equivalents to the specific aspects of the invention described herein. While the invention has been described with reference to particular aspects and implementations, it will understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The implementation was chosen and described in order to best explain the principles of the invention and the practical application, and to enable others of ordinary skill in the art to understand the invention for various implementations with various modifications as are suited to the particular use contemplated.

SEQUENCES
SEQ ID NO: 1
GGGCGGGCGGG(2AP)GGG
SEQ ID NO: 2
CCCGC
SEQ ID NO: 3
CTGGAAGCGGG
SEQ ID NO: 4
TGGAAGCGGG
SEQ ID NO: 5
GGGNGGGNGGGNGGG
SEQ ID NO: 6
CCCTCCCGCCCGCCC
SEQ ID NO: 7
GGGCGGGCGGG(2AP)GGGCCCGCTTCCAG
SEQ ID NO: 8
GGGTGGGTGGGTGGG
SEQ ID NO: 9
GGCGAAGGTC
SEQ ID NO: 10
GGCGAAGGT
SEQ ID NO: 11
TTCCAG

### REFERENCES

1. DeLuca, M.; Shi, Z.; Castro, C. E.; Arya, G., Dynamic DNA nanotechnology: toward functional nanoscale devices. Nanoscale Horizons 2020, 5, 182-201.
2. Zhang, D. Y.; Seelig, G., Dynamic DNA nanotechnology using strand-displacement reactions. Nat Chem 2011, 3 (2), 103-13.
3. Shin, J. S.; Pierce, N. A., A synthetic DNA walker for molecular transport. J Am Chem Soc 2004, 126 (35), 10834-5.
4. Yin, P.; Choi, H. M.; Calvert, C. R.; Pierce, N. A., Programming biomolecular self-assembly pathways. Nature 2008, 451 (7176), 318-22.
5. Yin, P.; Yan, H.; Daniell, X. G.; Turberfield, A. J.; Reif, J. H., A unidirectional DNA walker that moves autonomously along a track. Angew Chem Int Ed Engl 2004, 43 (37), 4906-11.
6. Zhang, D. Y.; Turberfield, A. J.; Yurke, B.; Winfree, E., Engineering entropy-driven reactions and networks catalyzed by DNA. Science 2007, 318 (5853), 1121-5.
7. Chen, X.; Briggs, N.; McLain, J. R.; Ellington, A. D., Stacking nonenzymatic circuits for high signal gain. Proc Natl Acad Sci USA 2013, 110 (14), 5386-91.
8. Kankia, B. I., Self-dissociative primers for nucleic acid amplification and detection based on DNA quadruplexes with intrinsic fluorescence. Anal Biochem 2011, 409 (1), 59-65.
9. Gogichaishvili, S.; Johnson, J.; Gvarjaladze, D.; Lomidze, L.; Kankia, B., Isothermal amplification of DNA using quadruplex primers with fluorescent pteridine base analogue 3-methyl isoxanthopterin. Biopolymers 2014, 101 (6), 583-90.
10. Taylor, A.; Joseph, A.; Okyere, R.; Gogichaishvili, S.; Musier-Forsyth, K.; Kankia, B., Isothermal quadruplex priming amplification for DNA-based diagnostics. Biophys Chem 2013, 171, 1-8.
11. Mathias, J.; Okyere, R.; Lomidze, L.; Gvarjaladze, D.; Musier-Forsyth, K.; Kankia, B., Thermal stability of quadruplex primers for highly versatile isothermal DNA amplification. Biophys Chem 2014, 185, 14-8.
12. Kankia, B. I.; Marky, L. A., DNA, RNA, and DNA/RNA oligomer duplexes: a comparative study of their stability, heat, hydration and Mg(2+) binding properties. J Phys Chem. B 1999, 103, 8759-8767.
13. Marky, L. A.; Breslauer, K. J., Calculating thermodynamic data for transitions of any molecularity from equilibrium melting curves. Biopolymers 1987, 26, 1601-1620.
14. Johnson, J.; Okyere, R.; Joseph, A.; Musier-Forsyth, K.; Kankia, B., Quadruplex formation as a molecular switch to turn on intrinsically fluorescent nucleotide analogs. Nucleic Acids Res 2013, 41 (1), 220-8.
15. Kankia, B. I.; Marky, L. A., Folding of the thrombin aptamer into a G-quadruplex with Sr(2+): stability, heat, and hydration. J Am Chem Soc 2001, 123 (44), 10799-804.
16. Mergny, J. L.; Phan, A. T.; Lacroix, L., Following G-quartet formation by UV-spectroscopy. FEBS Lett 1998, 435 (1), 74-8.
17. Oyaghire, S. N.; Cherubim, C. J.; Telmer, C. A.; Martinez, J. A.; Bruchez, M. P.; Armitage, B. A., RNA G-Quadruplex Invasion and Translation Inhibition by Antisense gamma-Peptide Nucleic Acid Oligomers. Biochemistry 2016, 55 (13), 1977-88.

1. A method for detecting a target nucleic acid, the method comprising:
obtaining a sample, the sample comprising the target nucleic acid having a target sequence;
providing an oligonucleotide, the oligonucleotide comprising a first nucleotide sequence comprising a quadruplex-forming sequence and a second nucleotide sequence operably linked to the first nucleotide sequence, the second nucleotide sequence being sufficiently complementary to the target sequence to hybridize therewith;
binding the target sequence to the second nucleotide sequence, thereby inducing the quadruplex-forming sequence to take a quadruplex conformation; and
detecting the oligonucleotide in the quadruplex conformation.

2. The method of claim 1, wherein the quadruplex-forming sequence comprises the formula G₃₊N₁₋₇G₃₊N₁₋₇G₃₊X₁₋₇G₀₋₃, the G content of the quadruplex-forming sequence is equal to or greater than 70%, N is any nucleotide, and X is a labeling compound or any nucleotide.

3. The method of claim 1, wherein the quadruplex-forming sequence comprises GGGCGGGCGGG(2AP)GGG (SEQ ID NO: 1).

4. The method of claim 1, further comprising forming a hairpin conformation with the oligonucleotide prior to binding the target sequence, wherein forming a hairpin conformation comprises hybridizing a stem sequence of the second nucleotide to the quadruplex-forming sequence of the first nucleotide sequence.

5. The method of claim 4, further comprising stabilizing the hairpin configuration with cesium ions, magnesium ions, or both.

6. The method of claim 4, wherein binding the target sequence to the second nucleotide sequence comprises first binding the target sequence to a toehold sequence that is operably linked to the stem sequence while the oligonucleotide is in the hairpin conformation.

7. The method of claim 4, wherein binding the target sequence to the second nucleotide sequence further comprises binding the target sequence to the stem sequence while the quadruplex-forming sequence dissociates from the stem sequence.

8. The method of claim 4, wherein the stem sequence comprises the formula C₃₊N₁₋₇ and N is any nucleotide.

9. (canceled)

10. The method of claim 4, further comprising heating the sample, thereby inducing the oligonucleotide to revert from the quadruplex conformation to the hairpin conformation.

11. The method of claim 1, further comprising stabilizing the quadruplex conformation with potassium ions.

12. (canceled)

13. (canceled)

14. The method of claim 1, wherein detecting the oligonucleotide in the quadruplex conformation further comprises registering a change in an optically detectable property.

15. (canceled)

16. (canceled)

17. The method of claim 1, wherein the method is performed without enzymes.

18. The method of claim 1, further comprising quantifying the target nucleic acid.

19. An oligonucleotide for use in binding a target nucleic acid in a non-enzymatic nucleic acid detection assay, the oligonucleotide comprising:
a first nucleotide sequence comprising a quadruplex-forming sequence; and
a second nucleotide sequence operably linked to the first nucleotide sequence;
wherein the quadruplex-forming sequence comprises the formula G₃₊N₁₋₇G₃₊N₁₋₇G₃₊X₁₋₇G₀₋₃, the G content of the quadruplex-forming sequence is equal to or greater than 70%, N is any nucleotide, and X is a detectable labeling compound or any nucleotide;
wherein the second nucleotide sequence comprises a stem sequence and a toehold sequence, the stem sequence being sufficiently complementary to a portion of the quadruplex-forming sequence to hybridize therewith and sufficiently complementary to a portion of the target nucleic acid to hybridize therewith, the toehold sequence being sufficiently complementary to the target nucleic acid to hybridize therewith; and
wherein the stem sequence comprises the formula C₃₊N₁₋₇.

20. (canceled)

21. (canceled)

22. The oligonucleotide of claim 19, wherein the oligonucleotide takes a hairpin conformation under a first set of conditions, and wherein the first nucleotide sequence of the oligonucleotide takes a quadruplex-conformation under a second set of conditions.

23. The oligonucleotide of claim 22, wherein the first set of conditions comprises less potassium in the surrounding environment than the second set of conditions.

24. The oligonucleotide of claim 22, wherein the first set of conditions comprises more cesium in the surrounding environment than the second set of conditions.

25. The oligonucleotide of claim 19, wherein the quadruplex-forming sequence comprises at least 70% sequence identity to GGGCGGGCGGG(2AP)GGG (SEQ ID NO: 1).

26. The oligonucleotide of claim 19, wherein the stem sequence comprises at least 70% sequence identity to CCCGC (SEQ ID NO: 2).

27. A method of diagnosing a subject with a disease or a disorder, making a prognosis of a disease or disorder, or predicting the development of a future disease or disorder, by detecting a target nucleic acid, the method comprising:
obtaining a bodily sample from the subject,
extracting nucleic acids from the bodily sample,
contacting the nucleic acids from the bodily sample with an oligonucleotide, the oligonucleotide comprising a first nucleotide sequence comprising a quadruplex-forming sequence and a second nucleotide sequence operably linked to the first nucleotide sequence, the second nucleotide sequence being sufficiently complementary to a target nucleic acid sequence to hybridize therewith;
if the target nucleic acid is present within the nucleic acids from the bodily sample, binding the target nucleic acid sequence to the second nucleotide sequence, thereby inducing the oligonucleotide to take a quadruplex conformation;
detecting the oligonucleotide in the quadruplex conformation; and
correlating the detection of the quadruplex conformation with a diagnosis of the disease or disorder, a prognosis of the disease or disorder, or the development of the disease or disorder.

28. (canceled)

29. (canceled)


SEQUENCE LISTING (11 sequences)

SEQ ID NO: 1 - DNA, 15 bp, Artificial Sequence
  synthetic construct
  misc_feature (12)..(12): a detection label
gggcgggcgggnggg

SEQ ID NO: 2 - DNA, 5 bp, Artificial Sequence
  synthetic construct
cccgc

SEQ ID NO: 3 - DNA, 11 bp, Artificial Sequence
  synthetic construct
ctggaagcggg

SEQ ID NO: 4 - DNA, 10 bp, Artificial Sequence
  synthetic construct
tggaagcggg

SEQ ID NO: 5 - DNA, 15 bp, Artificial Sequence
  synthetic construct
  misc_feature (4)..(4): n is a, c, g, or t
  misc_feature (8)..(8): n is a, c, g, or t
  misc_feature (12)..(12): n is a, c, g, or t
gggngggngggnggg

SEQ ID NO: 6 - DNA, 15 bp, Artificial Sequence
  synthetic construct
ccctcccgcccgccc

SEQ ID NO: 7 - DNA, 26 bp, Artificial Sequence
  synthetic construct
  misc_feature (12)..(12): a detection label
gggcgggcgggngggcccgcttccag

SEQ ID NO: 8 - DNA, 15 bp, Artificial Sequence
  synthetic construct
gggtgggtgggtggg

SEQ ID NO: 9 - DNA, 10 bp, Artificial Sequence
  synthetic construct
ggcgaaggtc

SEQ ID NO: 10 - DNA, 9 bp, Artificial Sequence
  synthetic construct
ggcgaaggt

SEQ ID NO: 11 - DNA, 6 bp, Artificial Sequence
  synthetic construct
ttccag